(19) United States
(12) Patent Application Publication
(10) Pub. No.: US 2024/0054907 A1
(43) Pub. Date: Feb. 15, 2024

(54) METHOD, DEVICE AND COMPUTER PROGRAM FOR PROVIDING HEARING ABILITY LEVEL ASSESSMENT AND AUDITORY TRAINING SERVICE

(71) Applicant: OLIVE UNION, INC., Seoul (KR)

(72) Inventors: Myunggeun SONG, Seoul (KR); Taeho SON, Seoul (KR); Jiwon CHOI, Seoul (KR)

(21) Appl. No.: 17/938,510

(22) Filed: Oct. 6, 2022

(30) Foreign Application Priority Data
Aug. 12, 2022 (KR) 10-2022-0101091

(51) Int. Cl. G09B 5/06; G06F 3/16; A61B 5/12

(52) U.S. Cl. CPC G09B 5/06; G06F 3/167; A61B 5/123

(57) ABSTRACT

Disclosed are a method, a device and a computer program for providing hearing level assessment and an auditory training service, the computer device. The method of providing hearing level assessment and an auditory training service includes providing information on a reference syllable to a user, simultaneously reproducing a plurality of syllables at a plurality of frequencies through a plurality of visual objects, receiving a selection input for any one visual object of the plurality of visual objects from the user, determining whether the any one visual object for which the selection input has been generated matches a specific visual object for which the reference syllable has been reproduced at a reference frequency, and assessing a hearing ability level and a speech discrimination level of the user for the reference frequency based on a result of the determination.

### CROSS-REFERENCE TO RELATED APPLICATIONS

A claim for priority under 35 U.S.C. § 119 is made to Korean Patent Application No. 10-2022-0101091 filed on Aug. 12, 2022, in the Korean Intellectual Property Office, the entire contents of which are hereby incorporated by reference.

### BACKGROUND

Embodiments disclosed herein relate to technology for a method, a device, and a computer program for providing hearing ability level assessment and an auditory training service.

People with hearing loss have great difficulty in communication in social life, and when communication is not conducted smoothly for a long period of time, may be exposed to mental illnesses such as depression and alienation.

Accordingly, people who are hard of hearing needs to receive hearing rehabilitation to be performed for the people who are hard of hearing to minimize the difficulties that may be caused by hearing loss.

For hearing rehabilitation, the hearing ability level of people who are hard of hearing should be accurately assessed, and auditory training to improve the hearing ability level should be provided.

However, currently, as described in Korean Patent No. 10-1420057, a technology for performing a speech perception test suitable for the development and level of infants and children has been proposed, but a technology for assessing the hearing ability level of people with hearing loss and providing auditory training have not been proposed.

Therefore, there is a need to provide a technique for assessing a hearing ability level and providing auditory training.

### SUMMARY

Embodiments of the inventive concept provide a method, a device and a computer program for assessing a user's hearing ability level and providing auditory training.

Further, embodiments of the inventive concept provide a method, a device, and a computer program for assessing a user's speech discrimination level and providing speech discrimination training.

However, the technical problems to be solved by inventive concept are not limited to the above problems, and may be variously expanded without departing from the technical spirit and scope of inventive concept.

According to an exemplary embodiment, a method of providing hearing level assessment and an auditory training service, the method being executed by a computer device including at least one processor, includes providing information on a reference syllable to a user, simultaneously reproducing a plurality of syllables at a plurality of frequencies through a plurality of visual objects, receiving a selection input for any one visual object of the plurality of visual objects from the user, determining whether any one visual object for which the selection input has been generated matches a specific visual object for which the reference syllable has been reproduced at a reference frequency, and assessing a hearing ability level and a speech discrimination level of the user for the reference frequency based on a result of the determination.

The simultaneously reproducing may include reproducing the reference syllable with the reference frequency through the specific visual object among the plurality of visual objects, and at the same time, reproducing at least one other syllable different from the reference syllable with at least one other frequency different from the reference frequency through at least one visual object other than the specific visual object among the plurality of visual objects.

The providing, the simultaneously reproducing, the receiving, the determining, and the assessing may be repeatedly performed as the reference frequency is sequentially changed to any one of a plurality of frequencies.

The providing, the simultaneously reproducing, the receiving, the determining, and the assessing may be repeatedly performed as the reference syllable is sequentially changed to any one of a plurality of syllables.

The method provides auditory training and speech discrimination training by repeatedly performing the providing, the simultaneously reproducing, the receiving, the determining, and the assessing.

The assessing includes at least one of reporting the hearing ability level of the user for each of the plurality of frequencies to the user, or reporting the speech discrimination level of the user for each of the plurality of syllables to the user.

The method may further include determining the reference syllable and the reference frequency using an artificial intelligence model learned in advance for the user.

The assessing may include reporting the hearing ability level and speech discrimination level of the user for the reference frequency to the user.

The assessing may include providing the user with a reward corresponding to the hearing ability level and speech discrimination level of the user for the reference frequency.

According to an exemplary embodiment, a computer-readable recording medium records a computer program causing computer device to execute a method of providing hearing level assessment and an auditory training service, the method including providing information on a reference syllable to a user, simultaneously reproducing a plurality of syllables at a plurality of frequencies through a plurality of visual objects, receiving a selection input for any one visual object of the plurality of visual objects from the user, determining whether the any one visual object for which the selection input has been generated matches a specific visual object for which the reference syllable has been reproduced at a reference frequency, and assessing a hearing ability level and a speech discrimination level of the user for the reference frequency based on a result of the determination.

According to an exemplary embodiment, a computer device for executing a method of providing hearing level assessment and an auditory training service includes at least one processor configured to execute computer readable instructions, wherein the at least one processor includes a providing unit configured to provide information on a reference syllable to a user, a reproducing unit configured to simultaneously reproduce a plurality of syllables at a plurality of frequencies through a plurality of visual objects, a receiving unit configured to receive a selection input for any one of the plurality of visual objects from the user, a determining unit configured to determine whether any one visual object for which the selection input has been generated matches a specific visual object for which the reference syllable has been reproduced at a reference frequency, and an assessing unit configured to assess a hearing ability level and speech discrimination level of the user for the reference frequency based on a result of the determination.

According to an exemplary embodiment, a method of providing hearing ability level assessment and an auditory training service, the method being executed by a computer device including at least one processor, includes reproducing a unique sound corresponding to a specific visual object at a specific frequency corresponding to the specific visual object through the specific visual object among a plurality of visual objects, receiving a selection input for any one of the plurality of visual objects from the user, determining whether the any one visual object for which the selection input has been generated matches the specific visual object for which the unique sound has been reproduced, and assessing a hearing ability level of the user for the specific frequency based on a result of the determination.

Each of the plurality of visual objects may enable a unique sound corresponding to each of the plurality of visual objects to be reproduced at a frequency corresponding to each of the plurality of visual objects.

Each of the plurality of visual objects may enable a unique sound corresponding to each of the plurality of visual objects to be reproduced at a frequency corresponding to each of the plurality of visual objects.

The method provides auditory training by repeatedly performing the reproducing, the receiving, the determining, and the assessing.

The method may further include reporting the hearing ability level of the user for frequencies respectively corresponding to the plurality of visual objects to the user.

The method may further include providing the user with a reward corresponding to the hearing ability level of the user for the specific frequency.

According to an exemplary embodiment, a computer-readable recording medium records a computer program causing computer device to execute a method of providing hearing level assessment and an auditory training service, the method including reproducing a unique sound corresponding to a specific visual object at a specific frequency corresponding to the specific visual object through the specific visual object among a plurality of visual objects, receiving a selection input for any one of the plurality of visual objects from the user, determining whether the any one visual object for which the selection input has been generated matches the specific visual object for which the unique sound has been reproduced, and assessing a hearing ability level of the user for the specific frequency based on a result of the determination.

According to an exemplary embodiment, a computer device for executing a method of providing hearing level assessment and an auditory training service includes at least one processor configured to execute computer readable instructions, wherein the at least one processor includes a reproducing unit configured to reproduce a unique sound corresponding to a specific visual object at a specific frequency corresponding to the specific visual object through the specific visual object among a plurality of visual objects a receiving unit configured to receive a selection input for any one of the plurality of visual objects from the user, a determining unit configured to determine whether the any one visual object for which the selection input has been generated matches the specific visual object for which the unique sound has been reproduced, and an assessing unit configured to assess a hearing ability level of the user for the specific frequency based on a result of the determination.

### BRIEF DESCRIPTION OF THE FIGURES

The above and other objects and features will become apparent from the following description with reference to the following figures, wherein like reference numerals refer to like parts throughout the various figures unless otherwise specified, and wherein:

FIG. 1 is a diagram illustrating an example of a network environment according to an embodiment;

FIG. 2 is a block diagram illustrating an example of a computer device according to an embodiment;

FIG. 3 is a block diagram illustrating an example of components which may be included in a processor shown in FIG. 2;

FIG. 4 is a flowchart illustrating an example of a method for providing hearing ability level assessment and an auditory training service that the computer device shown in FIG. 2 is able to perform;

FIGS. 5A to 5D are diagrams illustrating screens of a user terminal to describe a method for providing hearing ability level assessment and an auditory training service illustrated in FIG. 4;

FIG. 6 is a block diagram illustrating another example of components which may be included in a processor shown in FIG. 2;

FIG. 7 is a flowchart illustrating another example of a method for providing hearing ability level assessment and an auditory training service that the computer device shown in FIG. 2 is able to perform; and

FIGS. 8A to 8B are diagrams illustrating screens of a user terminal to describe a method for providing hearing ability level assessment and an auditory training service illustrated in FIG. 7.

### DETAILED DESCRIPTION

Hereinafter, embodiments of the inventive concept will be described in detail with reference to the exemplary drawings. However, it will be understood that the inventive concept is by no means restricted or limited in any manner by these embodiments. In addition, the same reference numeral shown in each drawing indicates the same component.

In addition, terminologies used in the present specification are used to properly express preferred embodiments of the inventive concept, and may be changed depending on the intention of viewers or operators, or customs in the field to which the inventive concept belongs. Accordingly, definitions of these terminologies should be made based on the content throughout this specification. For example, the singular expressions include plural expressions unless the context clearly dictates otherwise. Also, in this specification, the terms “comprises” and/or “comprising” are intended to specify the presence of stated features, integers, steps, operations, elements, parts or combinations thereof, but do not preclude the presence or addition of steps, operations, elements, parts, or combinations thereof.

Also, it should be understood that various embodiments of inventive concept are different from each other but are not necessarily mutually exclusive. For example, specific shapes, structures, and characteristics described herein may be implemented in other embodiments without departing from the spirit and scope of inventive concept in relation to one embodiment. In addition, it should be understood that the position, arrangement, or configuration of individual components may be changed in each embodiment without departing from the spirit and scope of inventive concept.

In the following embodiments, a method for providing hearing ability level assessment and an auditory training service performed by a system for providing hearing ability level assessment and an auditory training service will be described. As a result of performing the method for providing hearing ability level assessment and an auditory training service, the hearing ability level assessment and auditory training service may be provided to the user through a user terminal.

A system for providing hearing ability level assessment and an auditory training service according to an embodiment may be implemented by at least one computer device implementing a server or an electronic device (e.g., a user terminal) to be described later. A computer program according to an embodiment may be installed and driven in the computer device, and the computer device may perform a method for providing hearing ability level assessment and an auditory training service according to an embodiment under the control of the driven computer program. The above-described computer program may be stored in a computer-readable recording medium in combination with a computer device to allow the computer device to execute a method for providing hearing ability level assessment and an auditory training service. The computer program described herein may have the form of one independent program package, or the form of one independent program package may be pre-installed in a computer device and linked with an operating system or other program packages.

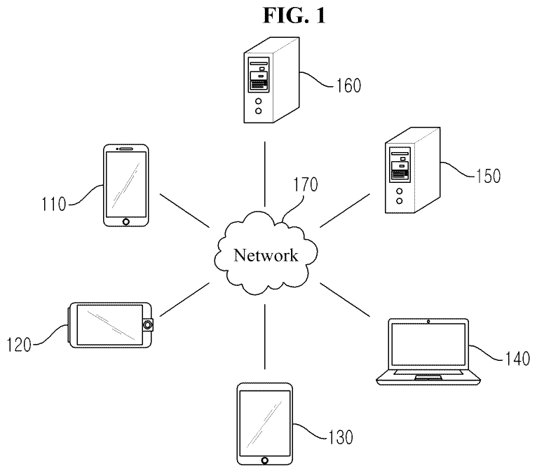

FIG. 1 is a diagram illustrating an example of a network environment according to an embodiment. The network environment of FIG. 1 shows an example including a plurality of electronic devices 110, 120, 130, and 140, a plurality of servers 150 and 160, and a network 170.

FIG. 1 is an example for describing the inventive concept, and the number of electronic devices or the number of servers is not limited as in FIG. 1. In addition, the network environment of FIG. 1 is only for describing one example of environments applicable to the present embodiments, and the environment applicable to the present embodiments is not limited to the network environment of FIG. 1.

The plurality of electronic devices 110, 120, 130, and 140 may be a fixed terminal implemented as a computer device or a mobile terminal. Examples of the plurality of electronic devices 110, 120, 130, and 140 include a smart phone, a mobile phone, a navigation device, a computer, a notebook computer, a digital broadcasting terminal, a personal digital assistant (PDA), a portable multimedia player (PMP), and a tablet PC. For example, although a smartphone is shown in FIG. 1 as an example of the electronic device 110, in embodiments, the electronic device 110 may refer to one of various physical computer devices capable of substantially communicating with other electronic devices 120, 130, and 140 and/or the server 150 or 160 through the network 170 using a wireless or wired communication method.

Hereinafter, the electronic devices 110, 120, 130, and 140 may refer to terminals of users receiving hearing ability level assessment and auditory training services.

The communication method is not limited, and may include not only a communication method using a communication network (e.g., a mobile communication network, a wired Internet, a wireless Internet, a broadcasting network) in which the network 170 may be included, but also short-range wireless communication between devices. For example, the network 170 may include one or more of networks, such as a personal area network (PAN), a local area network (LAN), a campus area network (CAN), a metropolitan area network (MAN), a wide area network (WAN), and a broadband network (BBN), the Internet, and the like. In addition, the network 170 may include any one or more of network topologies including a bus network, a star network, a ring network, a mesh network, a star-bus network, a tree or hierarchical network, and the like, but is not limited thereto.

Each of the servers 150 and 160 may communicate with the plurality of electronic devices 110, 120, 130, and 140 via the network 170 and may be implemented with a computer device or a plurality of computer devices that provides commands, codes, files, content, services, or the like. For example, the server 150 may be a system that provides a service to the plurality of electronic devices 110, 120, 130, and 140 connected through the network 170.

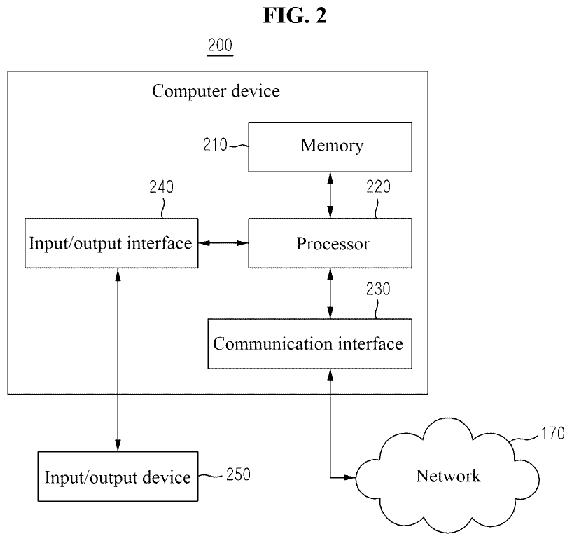

FIG. 2 is a block diagram illustrating an example of a computer device according to an embodiment. Each of the plurality of electronic devices 110, 120, 130, and 140 or each of the servers 150 and 160 which have been described above may be implemented by a computer device 200 illustrated in FIG. 2.

As shown in FIG. 2, the computer device 200 may include a memory 210, a processor 220, a communication interface 230, and an input/output interface 240. The memory 210 is a computer-readable recording medium and may include a random access memory (RAM), a read only memory (ROM), and a permanent mass storage device such as a disk drive. Here, a permanent mass storage device such as a ROM and a disk drive may be included in the computer device 200 as a separate permanent storage device that is distinct from the memory 210. Also, the memory 210 may store an operating system and at least one program code. These software components may be loaded into the memory 210 from a separate computer-readable recording medium distinct from the memory 210. The separate computer-readable recording medium may include a computer-readable recording medium such as a floppy drive, a disk, a tape, a DVD/CD-ROM drive, and a memory card. In another embodiment, the software components may be loaded into the memory 210 through the communication interface 230 rather than a computer-readable recording medium. For example, the software components may be loaded into the memory 210 of the computer device 200 based on a computer program installed by files received through the network 170.

The processor 220 may be configured to process instructions of a computer program by performing basic arithmetic, logic, and input/output operations. The instructions may be provided to the processor 220 by the memory 210 or the communication interface 230. For example, the processor 220 may be configured to execute a received instruction according to program codes stored in a recording device such as the memory 210.

The communication interface 230 may provide a function for allowing the computer device 200 to communicate with other devices (e.g., the storage devices described above) through the network 170. For example, a request, command, data, file, or the like generated by the processor 220 of the computer device 200 according to program codes stored in a recording device such as the memory 210 may be transmitted to other devices via the network 170. Conversely, a signal, command, data, file, or the like from another device may be received by the computer device 200 through the communication interface 230 of the computer device 200 via the network 170. A signal, command, data or the like received through the communication interface 230 may be transmitted to the processor 220 or the memory 210, and the file or the like may be stored in a storage medium (e.g., the permanent storage device described above) which may be further included in the computer device 200.

The input/output interface 240 may be means for interface with a input/output device 250. For example, the input device may include a device such as a microphone, keyboard, or mouse, and the output device may include a device such as a display or a speaker. As another example, the input/output interface 240 may be means for interface with a device in which an input function and an output function are integrated, such as a touch screen. The input/output device 250 may be configured as one device with the computer device 200.

Also, in other embodiments, the computer device 200 may include fewer or more components than those of FIG. 2. However, there is no need to clearly show most of the prior art components. For example, the computer device 200 may be implemented to include at least a part of the above-described input/output device 250 or may further include other components such as a transceiver and a database.

Hereinafter, specific examples of a method, a device, and a computer program for providing hearing ability level assessment and an auditory training service will be described.

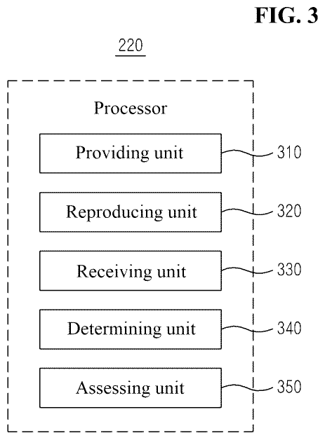

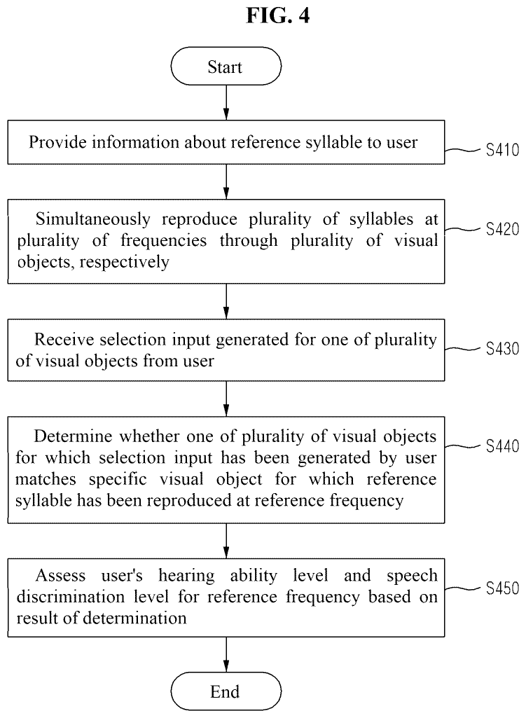

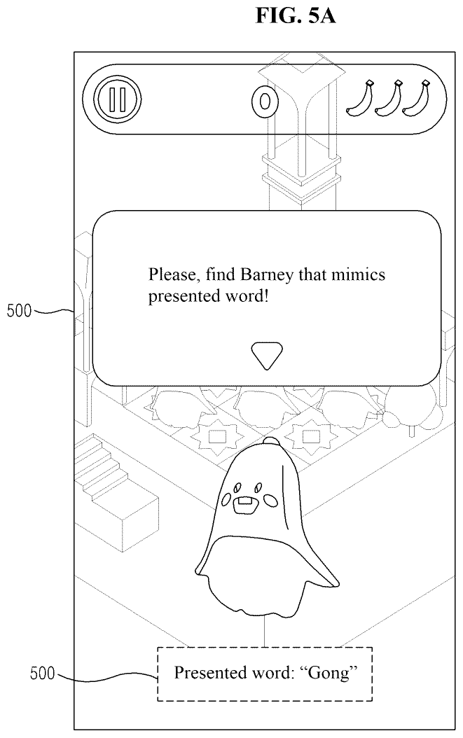

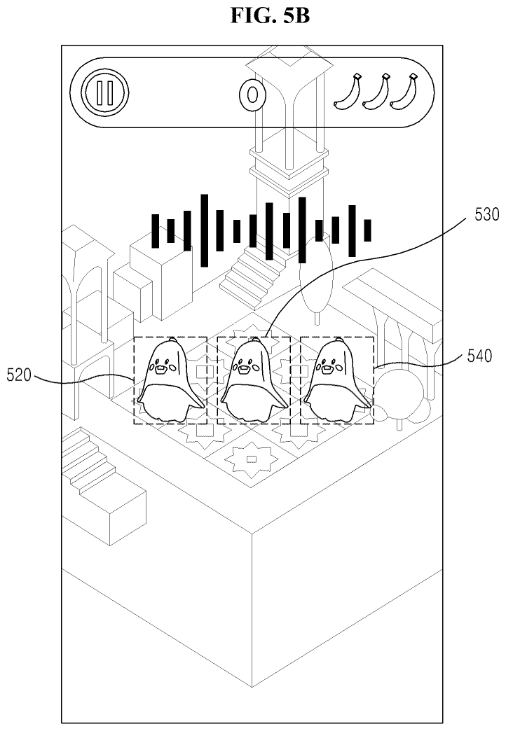

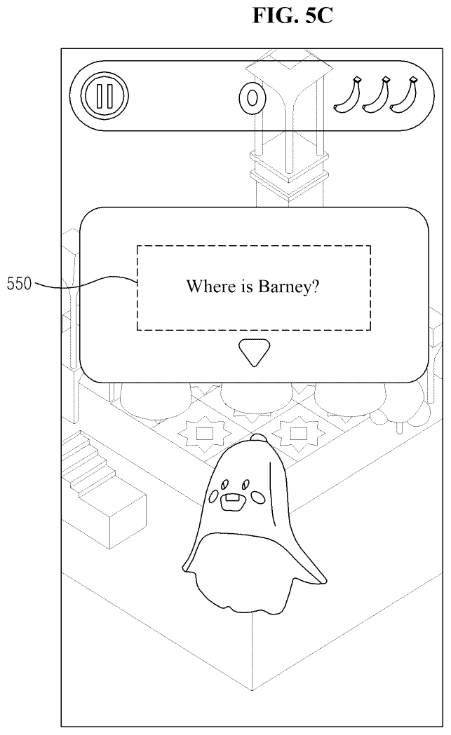

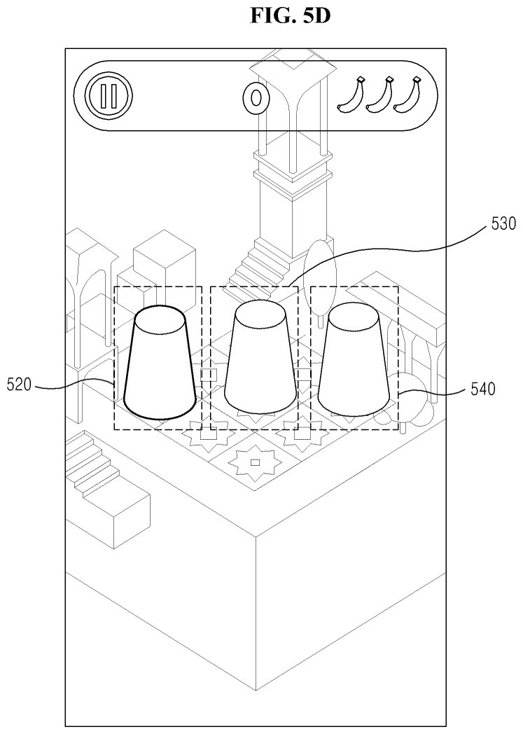

FIG. 3 is a block diagram illustrating an example of components which may be included in a processor shown in FIG. 2, FIG. 4 is a flowchart illustrating an example of a method for providing hearing ability level assessment and an auditory training service that the computer device shown in FIG. 2 is able to perform, and FIGS. 5A to 5D are diagrams illustrating screens of a user terminal to describe a method for providing hearing ability level assessment and an auditory training service illustrated in FIG. 4.

In the embodiments of inventive concept, the computer device 200 may provide a corresponding service to a user by performing a method for providing hearing ability level assessment and an auditory training service, which will be described later, in response to a service request generated by a user who wants to receive a hearing ability level assessment and the auditory training service through a dedicated application installed in a terminal (e.g., the electronic device 110) owned by the user or a server, or connection to a web/mobile site. To this end, the computer device 200 may be configured with a system for providing a hearing ability level assessment and an auditory training service, which is a subject that performs a method for providing a hearing ability level assessment and an auditory training service. For example, the system for providing a hearing ability level assessment and an auditory training service may be implemented in the form of a program that operates independently, or may be configured in the form of an in-app of a dedicated application to be able to operate on the dedicated application.

The processor 220 of the computer device 200 may be implemented as a component for performing the method for providing a hearing ability level assessment and an auditory training service according to FIG. 4. For example, the processor 220 may include a providing unit 310, a reproducing unit 320, a receiving unit 330, a determining unit 340, and an assessing unit 350 as show in in FIG. 3 to perform steps S410 to S450 shown in FIG. 4. According to embodiments, the components of the processor 220 may be selectively included in or excluded from the processor 220. Also, according to an embodiment, the components of the processor 220 may be separated from or merged into each other to express the functions of the processor 220.

The processor 220 and the components of the processor 220 may control the computer device 200 to perform steps S410 to S450 included in the method for providing a hearing ability level assessment and an auditory training service of FIG. 4. For example, the processor 220 and components of the processor 220 may be implemented to execute instructions according to the codes of an operating system included in the memory 210 and the codes of at least one program.

Here, the components of the processor 220 may be expressions of different functions performed by the processor 220 according to instructions provided by the program codes stored in the computer device 200. For example, the providing unit 310 may be used as a functional representation of the processor 220 that controls the computer device 200 to provide information on a reference syllable to the user.

The processor 220 may read a necessary instruction from the memory 210 in which instructions related to the control of the computer device 200 are loaded. In this case, the instruction which has been read may include an instruction for allowing the processor 220 to execute steps S410 to S450 to be described later.

Steps S410 to S450 to be described later may be performed in an order different from that shown in FIG. 4, and some of steps S410 to S450 may be omitted or additional processes may be further included.

In step S410, the processor 220 (more specifically, the providing unit 310 included in the processor 220) may provide information on a reference syllable to a user.

For example, the processor 220 may display information 510 on the reference syllable in a text format on a screen 500 of the user terminal as shown in FIG. 5A.

Here, the reference syllable may be determined as any one of syllables that the user has been unable to discriminate through a speech discrimination test performed on the user before step S410.

In addition, the reference syllable may be determined using an artificial intelligence model previously trained for the user. In this case, the artificial intelligence model may be trained in advance by using the results of the speech discrimination test performed on the user as training data, or by using the results of the method for providing a hearing ability level assessment and an auditory training service which has been previously performed as training data.

In step S420, the processor 220 (more specifically, the reproducing unit 320 included in the processor 220) may simultaneously reproduce a plurality of syllables at a plurality of frequencies through a plurality of visual objects.

In more detail, the processor 220 may reproduce the reference syllable at a reference frequency through a specific visual object among the plurality of visual objects, and at the same time, reproduce at least one other syllable different from the reference syllable with at least one other frequency different from the reference frequency through at least one visual object other than the specific visual object among the plurality of visual objects to simultaneously reproduce the plurality of syllables at the plurality of frequencies through the plurality of visual objects.

For example, as shown in FIG. 5B, the processor 220 may reproduce a reference syllable (e.g., “gong”) at a reference frequency through a first visual object 520 among the plurality of visual objects 520, 530, and 540, and at the same time, reproduce different syllables (e.g., “bong” and “dong”) different from the reference syllable at different frequencies other than the reference frequency through the second and third visual objects 530 and 540, respectively.

The above-described steps S410 to S420 may present a question for asking the user to select a visual object in which the reference syllable is reproduced at the reference frequency.

In this case, the reference frequency may be determined as any one of frequencies that the user has been unable to discriminate through the hearing ability level test performed on the user before step S420.

In addition, the reference frequency may be determined using an artificial intelligence model previously trained for the user. In this case, the artificial intelligence model may be trained in advance by using the results of the hearing ability level test performed on the user as training data, or by using the results of the method for providing hearing ability level assessment and an auditory training service which has been previously performed as training data.

In step S430, the processor 220 (more specifically, the receiving unit 330 included in the processor 220) may receive a selection input generated for any one of the plurality of visual objects from the user.

For example, in step S420, when a user who has heard a plurality of syllables which have been reproduced at a plurality of frequencies through a plurality of visual objects, respectively, may generate an input for selecting one visual object 520 among the plurality of visual objects 520, 530, and 540 as shown in FIG. 5D according to a command 550 to find the visual object in which the reference syllable provided in step S410 as shown in FIG. 5C has been reproduced, the processor 220 may receive a selection input of the user for any one visual object 520.

This step S430 may be to receive the user's answer to the question presented through steps S410 to S420.

In step S440, the processor 220 (more specifically, the determining unit 340 included in the processor 220) may determine whether the any one visual object on which the selection input has been generated by the user matches a specific visual object in which the reference syllable has been reproduced at a reference frequency.

For example, when the user generates a selection input for the first visual object 520 in which the reference syllable has been reproduced at the reference frequency as in the example described above, the processor 220 may determine that the visual object 520 on which the selection input has been generated matches the first visual object 520 in which the reference syllable has been reproduced at the reference frequency.

On the other hand, when the user generates a selection input for the second visual object 530 other than the first visual object 520, the processor 220 may determine that the visual object 530 on which the selection input has been generated does not match the first visual object 520 in which the reference syllable has been reproduced at the reference frequency.

That is, step S440 may be to determine whether the user's answer that is input through step S430 for the question presented through steps S410 to S420 is correct or not.

In step S450, the processor 220 (more specifically, the assessing unit 350 included in the processor 220) may assess the user's hearing ability level and speech discrimination level for the reference frequency based on a result of the determination.

For example, when it is determined that the visual object for which the selection input has been generated through step S440 matches the visual object in which the reference syllable is reproduced at the reference frequency, the processor 220 may assess the hearing ability level and speech discrimination level of the user for the reference frequency as being good.

On the other hand, when it is determined that the visual object for which the selection input has been generated through step S440 does not match the visual object in which the reference syllable is reproduced at the reference frequency, the processor 220 may assess the hearing ability level and speech discrimination level of the user for the reference frequency as being not good.

Although it has been described that there are only two cases including a case in which the user's hearing ability level and speech discrimination level for the reference frequency is assessed as being good and a case in which the user's hearing ability level and speech discrimination level for the reference frequency is assessed as being not good, the user's hearing ability level and speech discrimination level may be assessed using a score without being limited thereto.

The assessed hearing ability level and speech discrimination level of the user for the reference frequency may be reported to the user. For example, the report may be displayed on a screen of a user terminal in the format of a text indicating that the user's hearing ability level and speech discrimination level for the reference frequency are good.

In addition, in step S450, the processor 220 may assess the user's hearing ability level and speech discrimination level for the reference frequency, and at the same time provide a reward (e.g., points, gift certificate, etc.) corresponding to the assessed result to the user. In particular, the processor 220 may provide different rewards to the user according to results of the assessment of the user's hearing ability level and speech discrimination level for the reference frequency. As an example, the different rewards may be provided to the user by distinguishing the case in which the user's hearing ability level and speech discrimination level for the reference frequency is assessed as being good from the case in which the user's hearing ability level and speech discrimination level for the reference frequency is assessed as being not good.

Although it has been described in the method for providing hearing ability level assessment and an auditory training service that, in step S420, the hearing ability level for the reference frequency and the speech discrimination level for the reference syllable are simultaneously assessed as the plurality of syllables are reproduced at a plurality of frequencies through a plurality of visual objects, respectively, the present disclosure is not limited thereto, and the hearing ability level for the reference frequency and the speech discrimination level for the reference syllable may be separately assessed.

For example, by simultaneously reproducing a plurality of syllables at a single frequency through a plurality of visual objects in step S420, it is possible to assess only the user's speech discrimination level for a reference syllable at the single frequency.

In addition, the method of providing hearing ability level assessment and an auditory training service may provide auditory training and speech discrimination training for a reference syllable at a reference frequency to the user by repeatedly performing steps S410 to S450. Accordingly, the user's ability to discriminate the reference syllable at the reference frequency may be improved through auditory training and speech discrimination training.

In this case, when it is assessed that the user's hearing ability level and speech discrimination level are improved in the process of repeatedly performing steps S410 to S450, the processor 220 may reproduce the reference syllable together with other syllables simultaneously under the condition that actual environmental noise (e.g., street noise, TV noise, etc.) is reproduced at a volume lower than a syllable reproduction volume, rather than merely reproduce other syllables together with the reference syllable simultaneously, thereby increasing the difficulty in hearing and speech discrimination.

In this case, the above-described steps S410 to S450 may be repeatedly performed as the reference frequency is sequentially changed to any one of a plurality of frequencies. For example, while the reference syllable is fixed, steps S410 to S450 may be performed in a state in which the reference frequency is determined as a first frequency, and steps S410 to S450 may be performed in a state in which it is determined that the reference frequency is changed to the second frequency, and steps S410 to S450 may be performed in a state in which it is determined that the reference frequency is changed to the third frequency.

Accordingly, auditory training and speech discrimination training for a plurality of frequency-specific reference syllables may be provided to the user, thereby improving the user's ability to discriminate the plurality of frequency-specific reference syllables.

Also, steps S410 to S450 may be repeatedly performed as the reference syllable is sequentially changed to any one of the plurality of syllables. For example, while the reference frequency is fixed, steps S410 to S450 may be performed in a state in which the reference syllable is determined as a first syllable, and steps S410 to S450 may be performed in a state in which it is determined that the reference syllable is changed to the second syllable, and steps S410 to S450 may be performed in a state in which it is determined that the reference syllable is changed to the third syllable.

Accordingly, auditory training and speech discrimination training for a plurality of syllable-specific reference frequencies may be provided to the user, thereby improving the user's ability to discriminate the plurality of syllables at the reference frequency.

Although it is described that steps S410 to S450 are repeatedly performed in a case in which the reference frequency is sequentially changed to any one of a plurality of frequencies and a case in which the reference syllable is sequentially changed to any one of the plurality of syllables, the inventive concept is not limited thereto, and steps S410 to S450 may be repeatedly performed when the reference frequency is sequentially changed to any one of a plurality of frequencies and at the same time the reference syllable is sequentially changed to any one of the plurality of syllables.

As a result of repeatedly performing steps S410 to S450 as the reference frequency is sequentially changed to any one of the plurality of frequencies as described above, the user's hearing ability level and speech discrimination level for each of the plurality of frequencies may be reported to the user, and as a result of repeatedly performing steps S410 to S450 as the reference syllable is sequentially changed to any one of the plurality of syllables, the user's hearing ability level and speech discrimination level for each of the plurality of syllables may be reported to the user. Each report may be provided to the user in the assessment step (S450).

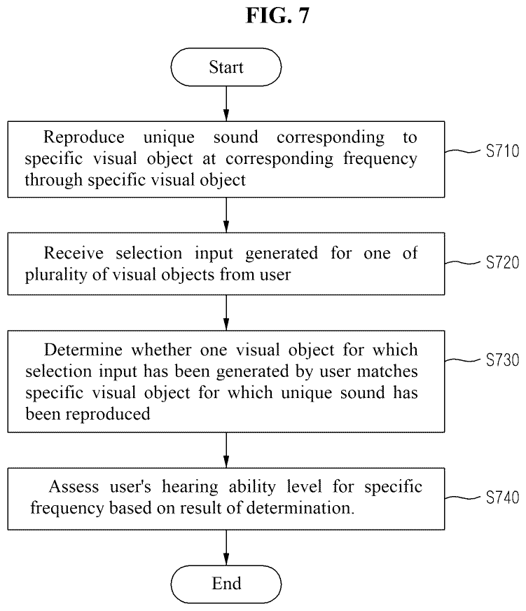

In addition, the processor 220 of the computer device 200 is not limited or limited to being implemented as a component for performing the method of providing hearing ability level assessment and an auditory training service according to FIG. 4 described, and may be implemented as a component for performing a method of providing hearing ability level assessment and an auditory training service according to FIG. 7. A detailed description thereof will be provided below.

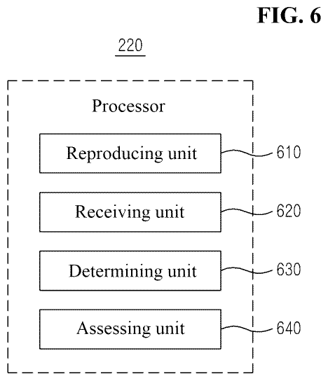

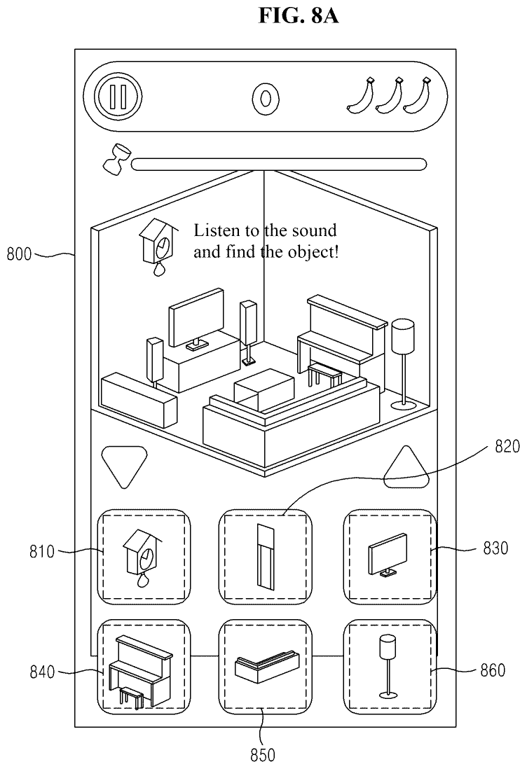

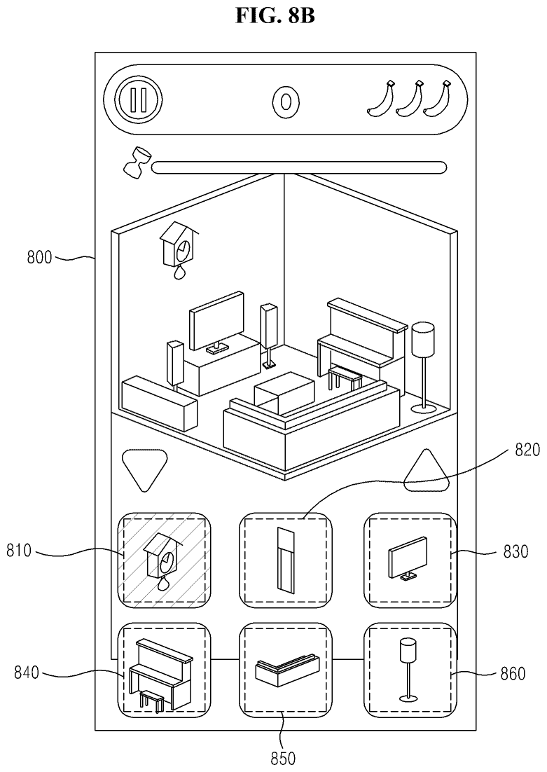

FIG. 6 is a block diagram illustrating an another example of components which may be included in a processor shown in FIG. 2, FIG. 7 is a flowchart illustrating another example of a method of providing hearing ability level assessment and an auditory training service that the computer device shown in FIG. 2 is able to perform, and FIGS. 8A to 8B are diagrams illustrating screens of a user terminal to describe a method for providing hearing ability level assessment and an auditory training service illustrated in FIG. 7.

Referring to FIGS. 6 to 7, the processor 220 may include a reproducing unit 610, a receiving unit 620, a determining unit 630, and an assessing unit 640 as show in in FIGS. 6 to perform steps S710 to S740 shown in FIG. 7. According to embodiments, the components of the processor 220 may be selectively included in or excluded from the processor 220. Also, according to an embodiment, the components of the processor 220 may be separated from or merged into each other to express the functions of the processor 220.

The processor 220 and the components of the processor 220 may control the computer device 200 to perform steps S710 to S740 included in the method for assessing the hearing ability level and providing an auditory training service of FIG. 6. For example, the processor 220 and the components of the processor 220 may be implemented to execute instructions according to the codes of an operating system included in the memory 210 and the codes of at least one program.

Here, the components of the processor 220 may be expressions of different functions performed by the processor 220 according to instructions provided by the program codes stored in the computer device 200. For example, the reproducing unit 610 may be used as a functional expression of the processor 220 that controls the computer device 200 to reproduce a unique sound corresponding to a specific visual object at a specific frequency through the specific visual object among a plurality of visual objects.

The processor 220 may read a necessary instruction from the memory 210 in which instructions related to the control of the computer device 200 are loaded. In this case, the instruction which has been read may include an instruction for allowing the processor 220 to execute steps S710 to S740 to be described later.

Steps S710 to S740 to be described later may be performed in an order different from that shown in FIG. 7, and some of steps S710 to S740 may be omitted or additional processes may be further included.

In step S710, the processor 220 (more specifically, the reproducing unit 610 included in the processor 220) may reproduce a unique sound corresponding to a specific visual object through the specific visual object among a plurality of visual objects at a specific frequency corresponding to the specific visual object.

For example, as shown in FIG. 8A, the processor 220 may reproduce a unique sound (e.g., the cuckoo sound of a cuckoo clock) corresponding to a specific visual object (e.g., 810) among a plurality of visual objects 810, 820, 830, 840, 850, and 860 displayed on a screen 800 of a user terminal at a specific frequency corresponding to the cuckoo sound, as shown in FIG. 8A.

In this case, each of the plurality of visual objects 810, 820, 830, 840, 850, and 860 may enable a unique sound corresponding to each of the plurality of visual objects 810, 820, 830, 840, 850, and 860 to be reproduced at a frequency corresponding to each of the visual objects. For example, the visual object 810 representing the cuckoo clock may enable the cuckoo sound of the cuckoo clock to be reproduced at the frequency of the cuckoo sound, the visual object 820 representing a window may enable a sound occurring in the process of opening and closing the window to be reproduced at the frequency of the sound occurring in the process of opening and closing the window, and the visual object 840 representing a piano may enable a piano sound to be reproduced at the frequency of the piano sound. Accordingly, the processor 220 may select a specific visual object from among the plurality of visual objects 810, 820, 830, 840, 850, and 860 in step S710, and reproduce only a unique sound of the specific visual object at the frequency of the specific visual object.

Here, the specific visual object may be determined to be a visual object for reproducing a unique sound having any one of frequencies which the user is unable to discriminate through the hearing ability level test performed on the user before step S710 among the plurality of visual objects 810, 820, 830, 840, 850, and 860.

In addition, the specific visual object may be determined using an artificial intelligence model previously learned for the user. In this case, the artificial intelligence model may be trained in advance by using the results of the hearing ability level test performed on the user as training data, or by using the results of the method for providing a hearing ability level assessment and an auditory training service which has been previously performed as training data.

The above-described step S710 may present a question for asking the user to select a visual object in which a unique sound is reproduced at the specific frequency.

In step S720, the processor 220 (more specifically, the receiving unit 620 included in the processor 220) may receive a selection input generated for any one of the plurality of visual objects from the user.

For example, in step S710, when the user generates an input for selecting one visual object 810 from among the plurality of visual objects 810, 820, 830, 840, 850, and 860 with respect to the unique sound of a specific visual object reproduced at a specific frequency of the specific visual object through the specific visual object 810 as shown in FIG. 8B, the processor 220 may receive the user's selection input for any one visual object 810.

Step S720 may be to receive the user's answer to the question presented through step S710.

In step S730, the processor 220 (more specifically, the determining unit 630 included in the processor 220) may determine whether the any one visual object on which the selection input has been generated by the user matches a specific visual object in which an unique sound has been reproduced.

For example, when the user generates a selection input for the visual object 810 indicating the cuckoo clock for which a unique sound has been reproduced at a corresponding frequency as in the example described above, the processor 220 may determine that the visual object 810 on which the selection input has been generated matches the visual object 810 indicating the cuckoo clock for which the unique sound has been reproduced at the corresponding frequency.

On the other hand, when the user generates a selection input for the visual object 840 indicating the piano rather than the visual object 810 indicating the cuckoo clock for which a unique sound has been reproduced at a corresponding frequency, the processor 220 may determine that the visual object 840 on which the selection input has been generated does not match the visual object 810 indicating the cuckoo clock for which the unique sound has been reproduced at a corresponding frequency.

That is, step S730 may be to determine whether the user's answer that is input through step S720 for the question presented through step S710 is correct or not.

In step S740, the processor 220 (more specifically, the assessing unit 640 included in the processor 220) may assess the user's hearing ability level for the specific frequency based on a result of the determination.

For example, in step S730, when it is determined that the visual object on which the selection input has been generated matches the visual object 810 indicating the cuckoo clock for which the unique sound has been reproduced at the corresponding frequency, in step S740, the processor 220 may assess the hearing ability level of the user for the specific frequency as being good.

On the other hand, in step S730, when it is determined that the visual object on which the selection input has been generated does not match the visual object 810 indicating the cuckoo clock for which the unique sound has been reproduced at the corresponding frequency, in step S740, the processor 220 may assess the hearing ability level of the user for the specific frequency as being not good.

Although it has been described that there are only two cases including a case in which the user's hearing ability level for the specific frequency is assessed as being good and a case in which the user's hearing ability level for the specific frequency is assessed as being not good, the user's hearing ability level may be assessed using a score without being limited thereto.

The assessed hearing ability level of the user for the specific frequency may be reported to the user. For example, the report may be displayed on a screen of a user terminal in the format of a text indicating that the user's hearing ability level for the specific frequency are good.

In addition, in step S740, the processor 220 may assess the user's hearing ability level for the specific frequency, and at the same time, provide a reward (e.g., points, gift certificate, etc.) corresponding to the assessed result to the user. In particular, the processor 220 may provide different rewards to the user according to results of the assessment of the user's hearing ability level for the specific frequency. As an example, the different rewards may be provided to the user by distinguishing the case in which the user's hearing ability level for the reference frequency is assessed as being good from the case in which the user's hearing ability level and speech discrimination level for the specific frequency is assessed as being not good.

Steps S710 to S740 described above may be repeatedly performed as a specific visual object is sequentially changed to any one of a plurality of visual objects. That is, as the specific visual object is sequentially changed to any one of the plurality of visual objects so as that a unique sound of each of the plurality of visual objects is reproduced at a frequency corresponding to each of the plurality of visual objects, steps S710 to S740 may be repeatedly performed.

Accordingly, hearing training for frequencies (a plurality of frequencies) respectively corresponding to the plurality of visual objects may be provided to the user, thereby enabling the user to improve a hearing ability level for each frequency.

In this case, when it is assessed that the user's hearing ability level is improved in the process of repeatedly performing steps S710 to S740, the processor 220 may reproduce a unique sound under the condition that actual environmental noise (e.g., street noise, etc.) is reproduced at a volume lower than a reproduction volume of the unique sound, rather than merely reproduce a unique sound corresponding a specific visual object at a specific frequency, thereby increasing the difficulty in hearing.

As a result of repeatedly performing steps S710 to S740 as the specific visual object is sequentially changed to any one of the plurality of visual objects, the user's hearing ability level for the frequency corresponding to each of the plurality of visual objects may be reported to the user. Each report may be provided to the user in the assessment step S740.

The apparatus described herein may be implemented with hardware components and software components and/or a combination of the hardware components and the software components. For example, the apparatus and components described in the embodiments may be implemented using one or more general-purpose or special purpose computers, such as a processor, a controller, an arithmetic logic unit (ALU), a digital signal processor, a microcomputer, a field programmable array (FPGA), a programmable logic unit (PLU), a microprocessor or any other device capable of executing and responding to instructions. The processing device may run an operating system (OS) and one or more software applications that run on the OS. The processing device also may access, store, manipulate, process, and create data in response to execution of the software. For convenience of understanding, one processing device is described as being used, but those skilled in the art will appreciate that the processing device includes a plurality of processing elements and/or multiple types of processing elements. For example, the processing device may include multiple processors or a single processor and a single controller. In addition, different processing configurations are possible, such a parallel processors.

The software may include a computer program, a piece of code, an instruction, or some combination thereof, for independently or collectively instructing or configuring the processing device to operate as desired. Software and/or data may be embodied in any type of machine, component, physical equipment, computer storage medium or device that is capable of providing instructions or data to or being interpreted by the processing device. The software also may be distributed over network coupled computer systems so that the software is stored and executed in a distributed fashion. In particular, the software and data may be stored by one or more computer readable recording mediums.

The above-described methods may be embodied in the form of program instructions that can be executed by various computer means and recorded on a computer-readable medium. In this case, the medium may be to continuously store the program executable by the computer, or to temporarily store the program for execution or download. In addition, the medium may be various recording means or storage means in the form of a single or several hardware combined, and is not limited to a medium directly connected to any computer system, and may exist distributed on a network. Examples of the media include magnetic media such as hard disks, floppy disks and magnetic tape, optical media such as CD-ROMs, DVDs, and magneto-optical medium such as floptical disks, ROM, RAM, flash memory, and the like, which is specifically configured to store and execute program instructions. In addition, examples of other media include recording media or storage media managed by an app store that distributes applications, sites that supply or distribute various other software, and servers.

Although the embodiments have been described by the limited embodiments and the drawings as described above, various modifications and variations are possible to those skilled in the art from the above description. For example, the described techniques may be performed in a different order than the described method, and/or components of the described systems, structures, devices, circuits, etc. may be combined or combined in a different form than the described method, or other components, or even when replaced or substituted by equivalents, an appropriate result can be achieved.

Therefore, other implementations, other embodiments, and equivalents to the claims are within the scope of the following claims.

The embodiments may propose a method, a device and a computer program for assessing a user's hearing ability level and providing auditory training.

In addition, the embodiments may propose a method, a device and a computer program for assessing a user's speech discrimination level and providing speech discrimination training.

However, the effects of the inventive concept are not limited to the above effects, and may be variously expanded without departing from the spirit and scope of the inventive concept.

While the inventive concept has been described with reference to exemplary embodiments, it will be apparent to those skilled in the art that various changes and modifications may be made without departing from the spirit and scope of the inventive concept. Therefore, it should be understood that the above embodiments are not limiting, but illustrative.

What is claimed is:

1. A method of providing hearing level assessment and an auditory training service, the method being executed by a computer device including at least one processor, the method comprising:
providing information on a reference syllable to a user;
simultaneously reproducing a plurality of syllables at a plurality of frequencies through a plurality of visual objects;
receiving a selection input for any one visual object of the plurality of visual objects from the user;
determining whether the any one visual object for which the selection input has been generated matches a specific visual object for which the reference syllable has been reproduced at a reference frequency; and
assessing a hearing ability level and a speech discrimination level of the user for the reference frequency based on a result of the determination.

2. The method of claim 1, wherein the simultaneously reproducing includes reproducing the reference syllable with the reference frequency through the specific visual object among the plurality of visual objects, and at the same time, reproducing at least one other syllable different from the reference syllable with at least one other frequency different from the reference frequency through at least one visual object other than the specific visual object among the plurality of visual objects.

3. The method of claim 1, wherein the providing, the simultaneously reproducing, the receiving, the determining, and the assessing are repeatedly performed as the reference frequency is sequentially changed to any one of a plurality of frequencies.

4. The method of claim 1, wherein the providing, the simultaneously reproducing, the receiving, the determining, and the assessing are repeatedly performed as the reference syllable is sequentially changed to any one of a plurality of syllables.

5. The method of claim 1, further comprising:
determining the reference syllable and the reference frequency using an artificial intelligence model learned in advance for the user.

6. The method of claim 1, wherein the assessing includes reporting the hearing ability level and speech discrimination level of the user for the reference frequency to the user.

7. The method of claim 1, wherein the assessing further includes providing the user with a reward corresponding to the hearing ability level and speech discrimination level of the user for the reference frequency.

8. A computer device for executing a method of providing hearing level assessment and an auditory training service, the computer device comprising;
at least one processor configured to execute computer readable instructions,
wherein the at least one processor includes:
a providing unit configured to provide information on a reference syllable to a user;
a reproducing unit configured to simultaneously reproduce a plurality of syllables at a plurality of frequencies through a plurality of visual objects;
a receiving unit configured to receive a selection input for any one visual object of the plurality of visual objects from the user;
a determining unit configured to determine whether the any one visual object for which the selection input has been generated matches a specific visual object for which the reference syllable has been reproduced at a reference frequency; and
an assessing unit configured to assess a hearing ability level and a speech discrimination level of the user for the reference frequency based on a result of the determination.

9. A method of providing hearing level assessment and an auditory training service, the method being executed by a computer device including at least one processor, the method comprising:
reproducing a unique sound corresponding to a specific visual object at a specific frequency corresponding to the specific visual object through the specific visual object among a plurality of visual objects;
receiving a selection input for any one visual object of the plurality of visual objects from a user;
determining whether the any one visual object for which the selection input has been generated matches the specific visual object for which the unique sound has been reproduced; and
assessing a hearing ability level of the user for the specific frequency based on a result of the determination.

10. The method of claim 9, wherein each of the plurality of visual objects enables a unique sound corresponding to each of the plurality of visual objects to be reproduced at a frequency corresponding to each of the plurality of visual objects.

11. The method of claim 9, wherein the reproducing, the receiving, the determining, and the assessing are repeatedly performed as the specific visual object is sequentially changed to any one of the plurality of visual objects.

12. The method of claim 11, the method provides auditory training by repeatedly performing the reproducing, the receiving, the determining, and the assessing.

13. The method of claim 11, the method provides reporting the hearing ability level of the user for frequencies respectively corresponding to the plurality of visual objects to the user.

14. The method of claim 9, further comprising:
providing the user with a reward corresponding to the hearing ability level of the user for the specific frequency.
